(19) United States
(12) Patent Application Publication
(10) Pub. No.: US 2023/0402137 A1
(43) Pub. Date: Dec. 14, 2023

(54) RETROSYNTHESIS AND PROXY CHEMICALS FOR LIFE-CYCLE ASSESSMENT

(71) Applicant: Microsoft Technology Licensing, LLC, Redmond, WA (US)

(72) Inventors: Jake Allen SMITH, Seattle, WA (US); Bichlien Hoang NGUYEN, Seattle, WA (US); Kali Diane FROST, Indianapolis, IN (US)

(73) Assignee: Microsoft Technology Licensing, LLC, Redmond, WA (US)

(21) Appl. No.: 17/804,551

(22) Filed: May 27, 2022

(51) Int. Cl. G16C 60/00; G16C 20/70; G16C 20/30

(52) U.S. Cl. CPC G16C 60/00; G16C 20/30; G16C 20/70

(57) ABSTRACT

A computing system is provided. The computing system comprises a processor, and memory comprising instructions executable by the processor to receive a chemical structure input, obtain retrosynthetic step data based on the chemical structure input, determine a chemical structure of a primary chemical in the retrosynthetic step data, the primary chemical being a chemical used as a starting material in a retrosynthetic step, when the structure of the primary chemical is not available in a life-cycle inventory (LCI), input the primary chemical into a trained proxy chemical selection model to select a proxy chemical for which an LCI is available, and obtain proxy chemical LCI data to include in an estimated LCI for a life cycle assessment (LCA).

### BACKGROUND

Life-cycle assessment (LCA) is a method for evaluating environmental impacts of a product throughout its entire life cycle. In LCA, production of a given product is broken into a series of process steps called unit processes. For each unit process, inputs such as materials and energy are quantified along with outputs such as products and waste. For many products, one or more of these unit processes involve synthetic chemicals. However, inputs and outputs for only a relatively small number of synthetic chemicals have been thoroughly quantified. As a result, LCA practitioners often estimate the inputs and outputs of non-qualified synthetic chemicals. One strategy for estimating inputs and outputs is the selection of proxy chemicals, which are chemicals present in an LCA database having similar structures to a synthetic chemical of interest. However, selection of proxy chemicals by LCA practitioners may be laborious and time-consuming. Furthermore, for a given synthetic chemical, selection of a proxy chemical may be variable, as selection is dependent upon the chemistry domain knowledge of LCA practitioners.

### SUMMARY

Examples are disclosed that relate to using retrosynthesis in estimating a life cycle inventory (LCI) for a life cycle analysis (LCA). One disclosed example provides a computing system. The computing system comprises a processor, and memory comprising instructions executable by the processor to receive a chemical structure input, obtain retrosynthetic step data based on the chemical structure input, and determine a chemical structure of a primary chemical in the retrosynthetic step data, the primary chemical being a chemical used as a starting material in a retrosynthetic step. The instructions are further executable to, when the structure of the primary chemical is not available in an LCI, input the primary chemical into a trained proxy chemical selection model to select a proxy chemical for which an LCI is available, and obtain proxy chemical LCI data to include in an estimated LCI for a life cycle assessment (LCA).

Another example provides a method for generating an estimated LCI for an LCA. The method comprises receiving a chemical structure input, obtaining retrosynthetic step data based on the chemical structure input, determining a chemical structure of a primary chemical in the retrosynthetic step data, the primary chemical being a chemical used as a starting material in a retrosynthetic step, and obtaining proxy chemical LCI data for the primary chemical to include in the estimated LCI for the LCA.

Another example provides a computing system comprising a processor, and memory comprising instructions executable by the processor to receive a chemical structure input, obtain retrosynthetic step data based on the chemical structure input, identify a chemical transformation in the retrosynthetic step data, retrieve LCI data associated with the chemical transformation; and include the LCI data in an estimated LCI for an LCA.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a block diagram depicting an example life-cycle assessment (LCA) comprising a plurality of life-cycle inventories (LCIs).

FIG. 2 shows example details of an LCI of FIG. 1.

FIG. 3 shows an example process flow for determining an estimated LCI.

FIG. 4 shows an example computing system with which the process flow of FIG. 3 may be implemented.

FIG. 5 shows a flow diagram depicting an example method for determining information an estimated LCI for an LCA.

FIG. 6 shows a block diagram of an example computing system.

### DETAILED DESCRIPTION

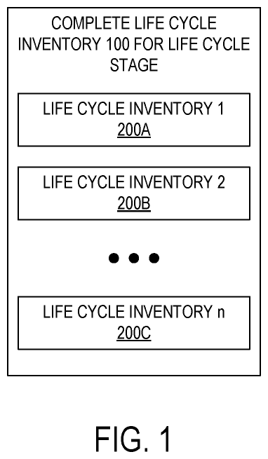

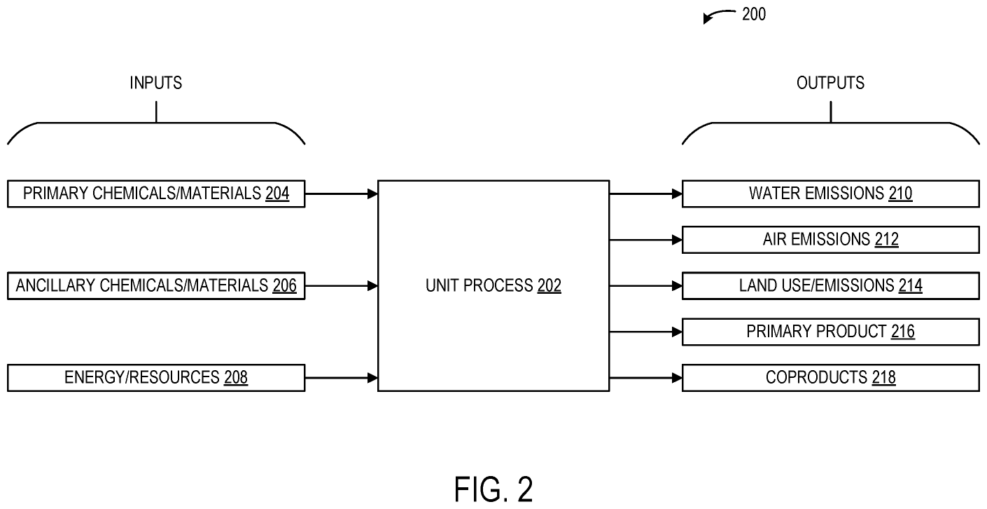

As described above, challenges may exist in accurately determining inputs and outputs for certain manufacturing processes for use in a life cycle inventory. FIG. 1 shows a block diagram depicting a collection of unit process life cycle inventories 200A-C (hereinafter LCIs 200A-C) for a life cycle stage. LCIs 200A-C may be summed to create a complete life cycle inventory 100 for the life cycle stage as a part of a life cycle assessment (LCA). As one example, LCIs 200A-C may represent manufacturing steps in a manufacturing process. FIG. 2 shows additional details of an LCI 200. LCI 200 may represent an estimated LCI as described below. LCI 200 includes inputs and outputs for a discrete unit process 202. Examples of unit processes 202 include manufacturing, mining, usage, transport, purification, refinement, and disposal. LCI 200 may represent any of LCI 1 200A, LCI 2 200B, and/or LCI n 200C. Examples of inputs into unit process 202 include primary chemicals and materials 204, ancillary chemicals and materials 206, and energy and resources 208. Examples of outputs from the unit process 202 include water emissions 210, air emissions 212, land use and emissions 214, a primary product 216, and coproducts 218. It will be appreciated that these inputs and outputs are presented for the purpose of example, and that any other suitable inputs and outputs may be included in the LCI 200.

As described above, inputs and outputs for a relatively small number of synthetic chemicals have been thoroughly quantified. As a result, the inputs and outputs of non-quantified chemicals are often estimated using proxy chemicals. However, selection of proxy chemicals by LCA practitioners may be laborious and time-consuming. Furthermore, for a given chemical, selection of a proxy chemical may vary from one LCA practitioner to another.

Accordingly, examples are disclosed that relate to the automated determination of an estimated LCI based upon the use of retrosynthetic data. The use of retrosynthetic data in estimating an LCI may allow proxy chemical selection and/or proxy transformation selection to be performed more quickly than manual methods and may help to reduce or eliminate variability in LCI estimation.

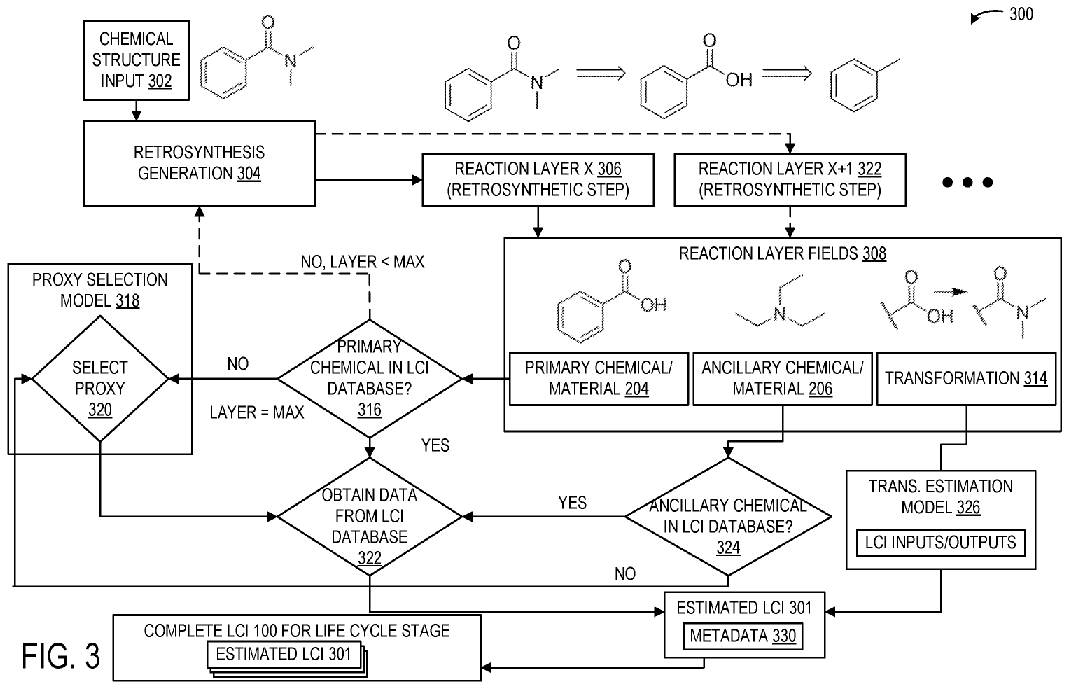

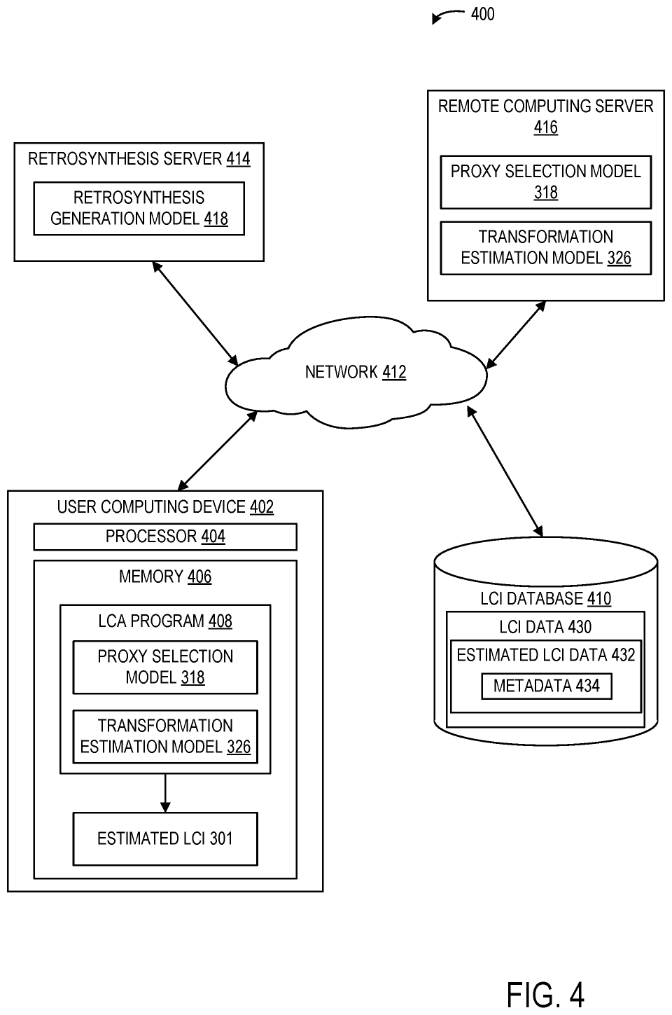

FIG. 3 shows a block diagram of an example process flow 300 for determining an estimated LCI 301, which is an example of LCI 200, for inclusion in an LCA as complete life cycle inventory 100 for a life cycle stage. The process flow 300 may utilize retrosynthetic analysis, machine learning-assisted proxy chemical selection, and/or machine learning-assisted transformation estimation in determining an LCI. The process flow 300 may be implemented by any suitable computing system. FIG. 4 shows one example of a suitable computing system 400 comprising a user computing device 402 including an LCA database 410, and an LCA program 408 configured to determine estimated LCIs using one or more of retrosynthetic analysis, machine-learning assisted proxy chemical selection, or machine-learning assisted transformation estimation. Other details of FIG. 4 are discussed in more detail below.

Process flow 300 first receives a chemical structure input 302. The chemical structure input 302 may comprise a structure drawn using a chemical structure drawing program, a chemical name, a unique chemical identifier such a Chemical Abstract Service (CAS) registry number or European Community (EC) number, a simplified molecular-input line-entry system (SMILES) string, or any other suitable input. The chemical structure input 302 corresponds to a chemical of interest, such as a chemical used in a manufactured good or in a chemical process. In this example, the chemical structure input 302 comprises N,N-dimethylbenzamide, although it will be appreciated that the LCA program 408 may accept any chemical structure input 302.

Through retrosynthesis generation 304, process flow 300 is configured to obtain retrosynthetic step data based on chemical structure input 302. The retrosynthetic step data in this example is shown as reaction layer X 306, a retrosynthetic step in which N,N-dimethylbenzamide is formed from benzoic acid. The retrosynthetic step data includes reaction layer fields 308 such as a primary chemical 204, an ancillary chemical 206, and a chemical transformation 314.

Primary chemical 204 comprises a chemical used as a starting material in the retrosynthetic step. In this example, the primary chemical 204 is benzoic acid, the ancillary chemical 206 is triethylamine, and the chemical transformation 314 is an amidation reaction.

When the structure of the primary chemical 204 is not available in the LCI database 410 and no retrosynthetic step data is available for the primary chemical 204 (NO, LAYER=MAX at 316), the LCA program 408 is configured to input the primary chemical 204 into a trained proxy chemical selection model 318 to select a proxy chemical (320) for which an LCI is available and obtain proxy chemical LCI data (322) to include in the estimated LCI 301. Proxy chemicals selected by the proxy chemical selection model 318 have LCIs 200 available in the LCI database 410 and are determined by the proxy chemical selection model 318 to be structurally similar to the primary chemical 204. Further details of the proxy selection model 318 are provided below in relation to description of FIG. 4. An advantage of selecting the proxy chemical 320 from the primary chemical 204 rather than selecting the proxy chemical 320 from the chemical structure input 302 is that the computing system 400 may be more likely to find suitably accurate LCI data.

On the other hand, when the structure of the primary chemical is not available in the LCI database 410 but retrosynthetic step data is available for the primary chemical 204, (NO, LAYER<MAX at 316) the LCA program 408 is configured to obtain retrosynthetic step data based on the chemical structure of the primary chemical 204, and determine a chemical structure of an additional primary chemical, namely, the primary chemical for retrosynthetic layer X+1 322, a retrosynthetic step in which benzoic acid is formed from toluene. Although not shown, an additional ancillary chemical (e.g. an oxidizing agent such as potassium permanganate) and an additional chemical transformation (e.g. an oxidation) are also determined in this example. While only two reaction layers are shown in FIG. 3, it will be appreciated that three, four, or any number of reaction layers may be generated. In some examples, reaction layers may be generated until either the primary chemical 204 is found in the LCI database 410 (YES at 316), or a maximum number of reaction layers is generated (NO, LAYER=MAX at 316). At 316, “LAYER=MAX” indicates that a reaction layer cannot be generated from the primary chemical 204, for example, because the primary chemical 204 may be structurally too simple for a viable chemical precursor to be available. Further, in some examples, a retrosynthesis algorithm may return a retrosynthesis tree comprising multiple retrosynthesis layers (e.g. all layers for a retrosynthesis in some examples). In such an example, rather than returning to the retrosynthesis algorithm to obtain a next layer of retrosynthesis step data upon finding that a chemical is not available in the LCI database 410, an additional primary and/or ancillary chemical may be obtained from the retrosynthesis data tree.

As described above, the LCA program 408 is further configured to determine a chemical structure of an ancillary chemical 206, if any, in the retrosynthetic step data. When the structure of the ancillary chemical 206 is available in the LCI database 410 (YES at 324), the LCA program is configured to obtain chemical LCI data (322) to include in the estimated LCI 301. LCA program 408 is further configured to, when the structure of the ancillary chemical 206 is not available in LCI database 410 (NO at 324), input the ancillary chemical 206 into the trained proxy chemical selection model 318 to obtain a proxy chemical for which an LCI is available, and obtain proxy chemical LCI data to include in the estimated LCI 301.

Continuing with FIG. 3, the LCA program 408 is further configured to identify a chemical transformation 314 in the retrosynthetic step data, retrieve LCI data associated with the chemical transformation 314, and include the LCI data associated with the chemical transformation 314 in the estimated LCI 301. Retrieving LCI data for the chemical transformation 314 may be performed by a trained transformation estimation model 326. An example trained transformation estimation model 326 is described in more detail below in relation to FIG. 4. Upon completion of the estimated LCI 301, the estimated LCI 301 may be included in the complete life cycle inventory 100, along with other LCIs in some examples.

In some examples, an estimated LCI 301 generated via process flow 300 may be stored in an LCI database (e.g. LCI database 410 of FIG. 4). This may allow estimated LCIs generated by process flow 300 to be retrieved for inclusion in other LCAs. Further, in some examples, metadata 330 related to the creation of an estimated LCI also may be stored for the estimated LCI. The metadata 330 may include, for example, information on how the estimated LCI was generated. Example information includes how many retrosynthetic steps were generated in a retrosynthesis before the chemical of the estimated LCI was output by the retrosynthetis algorithm, and how many proxy chemicals were selected per retrosynthesis step. Such data may be used, for example, to determine an uncertainty metric for the estimated LCI. The uncertainty metric may be represented as a score in some examples. In such an example, estimated LCIs may be rescored as the LCI database is updated with new data.

Turning now to FIG. 4, an example computing system 400 that may implement example process flow 300 is described in greater detail. The depicted computer system 400 is presented for the purpose of example, and any other suitable computer system may be used to implement process flow 300. In various examples, process flow 300 may be implemented by any suitable computing device or combination of computing devices of computing system 400.

Computing system 400 includes a user computing device 402, LCI database 410, a retrosynthesis server 414, and a remote computing server 416. User computing device 402 includes the processor 404 and memory 406 storing the LCA program 408. The proxy selection model 318 and the transformation estimation model 326 are executable by the LCA program 408 on the user computing device 402 in order to generate an estimated LCI, such as estimated LCI 301, for inclusion as complete life cycle inventory 100 for a life cycle stage in an LCA. Additionally or alternatively, the proxy selection model 318 and the transformation estimation model 326 may be executable by the remote computing server 416, and outputs of these models may be received by the user computing device 402.

The trained proxy chemical selection model 318 may comprise any suitable trained machine learning function. In some examples, the trained proxy chemical selection model may comprise an artificial neural network (ANN) that is trained with LCI data contained in a plurality of LCIs stored in LCI data. The LCI data includes, for each of the plurality of LCIs, a chemical structure of a chemical that the LCI describes. Such chemicals also may be referred to herein as possible proxy chemicals. The LCI data may be clustered in the trained proxy chemical selection model based at least upon similarities of the chemical structures of the possible proxy chemicals to one another. The chemical structure of a proxy chemical may be represented by a variety of methods, including a molecular graph in which nodes and edges represent atoms and bonds respectively, a SMILES string, or by a combination of the molecular graph and SMILES string. Other methods for representing a chemical structure of a proxy chemical include encoding the molecular graph M_{g} by a graph neural network (GNN) to output a high-level representation f_{g} or encoding the SMILES string Ms by a transformer to output a high-level representation fₛ. Clustering of the LCI data may be performed by K-Means, K-Medians, Mean-Shift clustering, or any other suitable clustering method. Clustering the LCI data in the trained proxy chemical selection model based at least upon the chemical structure of the proxy chemical may allow for suitably accurate selection of the proxy chemical.

Similarly, the transformation estimation model comprises any suitable trained machine learning function. In some examples, the transformation estimation model comprises an ANN that is trained with LCI data contained in a plurality of LCIs. The LCI data includes at least a starting material, a primary product, and an energy input. The LCI data further includes a reaction representation, the reaction representation being determined based upon a difference between the starting material and the primary product. The LCI data is clustered in the transformation estimation model 326 based at least upon the reaction representation. The reaction representation may be generated by a variety of methods, including a condensed graph of reaction (CGR), a SMILES Arbitrary Target Specification (SMARTS) string, or a combination of CGR and a SMARTS string. Other methods for generating the reaction representation include encoding the CGR R_{g} by a graph neural network (GNN) to output a high-level representation f_{g′} or encoding the SMARTS string Rs by a transformer to output a high-level representation f_{s′}. Clustering of the LCI data may be performed by K-Means, K-Medians, Mean-Shift clustering, or any other suitable clustering method. Clustering the LCI data in the transformation estimation model 326 based at least upon the reaction representation may allows for LCI data associated with the chemical transformation to be accurately selected.

Retrosynthesis server 414 executes a retrosynthesis generation model 418 that performs retrosynthesis generation 304 to generate the reaction layers and reaction layer fields 308. This may be accomplished by algorithms such as those used by commercially available retrosynthetic software. Examples include such software as SYNTHIA™ (MilliporeSigma, Burlington, MA, USA) and IBM RXN (International Business Machines Corporation, Armonk, New York, USA). In some examples, a retrosynthesis program may reside on user computing device 402.

The LCI database 410 includes LCI data 430 for potential proxy chemicals and is accessible by the LCA program of the user computing device 402. Potential proxy chemicals include chemicals for which LCI data has been determined, empirically or by other methods. LCI database 410 also may store estimated LCI data 432 comprising estimated LCIs that have been determined, for example, using process flow 300. In some such examples, metadata 434 for an estimated LCI comprising information on how the estimated LCI was determined also may be stored. Such metadata may include a score that represents an uncertainty metric in some examples. Additionally or alternatively, LCI data 430 may be stored in the memory 406 of the user computing device 402.

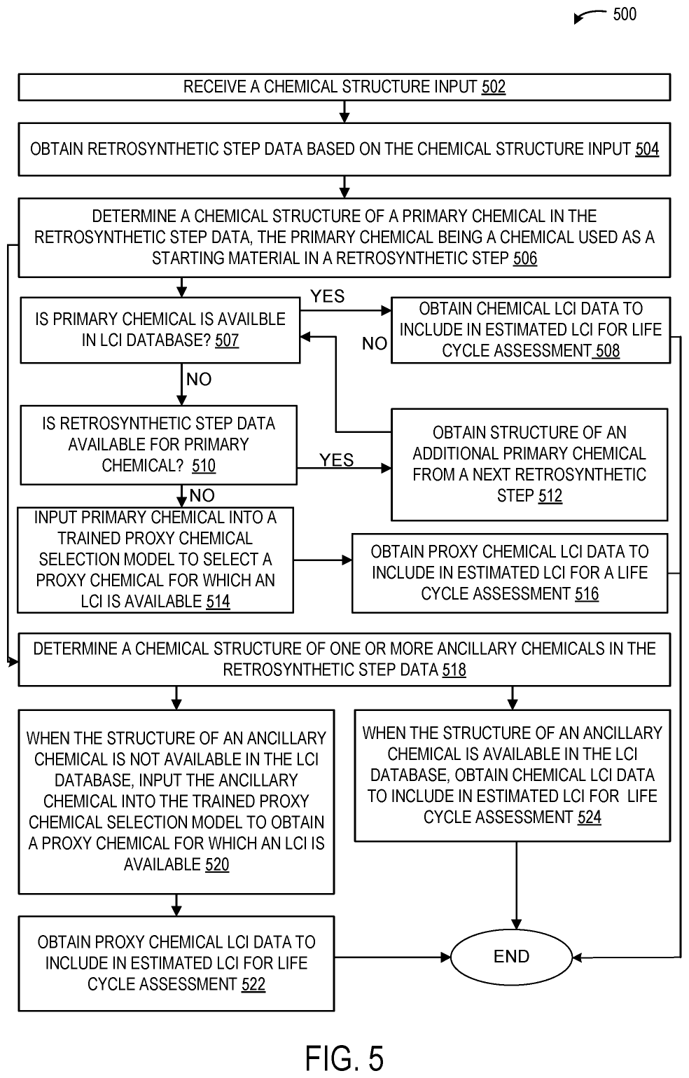

FIG. 5 shows a flow diagram depicting an example method 500 for generating an LCA. It will be appreciated that method 500 may be implemented using the above-described computing system 400 or other suitable hardware and software componentry. Method 500 may be used with a computing system comprising a processor, and memory comprising instructions executable by the processor. The following description of method 500 is provided by way of example and is not meant to be limiting. Therefore, it is to be understood that method 500 may include additional and/or alternative steps relative to those illustrated in FIG. 5. Further, it is to be understood that the steps of method 500 may be performed in any suitable order. Further still, it is to be understood that one or more steps may be omitted from method 500 without departing from the scope of this disclosure.

At 502, method 500 comprises receiving a chemical structure input. At 504, method 500 comprises obtaining retrosynthetic step data based on the chemical structure input. At 506, method 500 comprises determining a chemical structure of a primary chemical in the retrosynthetic step data, the primary chemical being a chemical used as a starting material in a retrosynthetic step. At 507, method 500 comprises determining whether an LCI for the primary chemical is available in a life-cycle inventory (LCI) database. If the primary chemical is available in the LCI database, then method 500 comprises, at 508, obtaining chemical LCI data to include in an estimated LCI for a life cycle assessment. In some examples, the LCI data may comprise a previously estimated LCI. In some such examples, metadata for the previously estimated LCI may be obtained to assess a quality and/or relevance of the previously estimated LCI.

On the other hand, if the primary chemical is not available in the LCI database, method 500 comprises determining whether there is retrosynthetic step data available for the primary chemical. If retrosynthetic step data is available for the primary chemical, then method 500 comprises, at 512, obtaining a structure of an additional primary chemical from a next retrosynthetic step and determining whether the additional primary chemical is available in the LCI database.

Processes 507, 510, and 512 may repeat until either the primary chemical is found in the LCI database, or a maximum number of reaction layers or a last retrosynthesis reaction layer is reached. (for example, when the primary chemical may be structurally too simple for a viable chemical precursor to be available).

If no retrosynthetic step data or LCI data is available for a primary chemical (whether an initial primary chemical from a first retrosynthetic step or an additional primary chemical from a later retrosynthetic step), then method 500 comprises, at 514, inputting the primary chemical into a trained proxy chemical selection model to select a proxy chemical for which an LCI is available. Further, at 516, method 500 comprises obtaining proxy chemical LCI data from the trained proxy chemical selection model to include in the estimated LCI for the life cycle assessment. In some examples, the trained proxy chemical selection model may comprise an artificial neural network (ANN) that is trained with LCI data contained in a plurality of LCIs. In other examples, the trained proxy chemical selected model may comprise any other suitable type of model. The LCI data may include, for each of the plurality of LCIs, a chemical structure of the possible proxy chemicals that are in the LCIs. The LCI data may be clustered in the trained proxy chemical selection model based at least upon the chemical structures of the possible proxy chemicals.

In some examples, an ancillary chemical also may be identified in retrosynthetic step data. As such, at 518, method 500 comprises determining a chemical structure of each of one or more ancillary chemicals in the retrosynthetic step data. When the structure of the ancillary chemical is not available in the LCI database, method 500 may comprise, at 520, inputting the ancillary chemical into the trained proxy chemical selection model to obtain a proxy chemical for which an LCI is available, and at 522, obtaining proxy chemical LCI data to include in the LCI for the life cycle assessment. On the other hand, at 524, when the structure of the ancillary chemical is available in the LCI database, method 500 comprises obtaining chemical LCI data to include in the LCI for the life cycle assessment.

By using a trained machine learning model comprising clustered possible proxy chemicals combined with retrosynthesis to identify a proxy chemical for another chemical for which LCI data is not available, an estimated LCI may be obtained more quickly and with less variability than via manual selection. This may help to perform LCAs more quickly and consistently compared to the manual identification of proxy chemicals.

In some embodiments, the methods and processes described herein may be tied to a computing system of one or more computing devices. In particular, such methods and processes may be implemented as a computer-application program or service, an application-programming interface (API), a library, and/or other computer-program product.

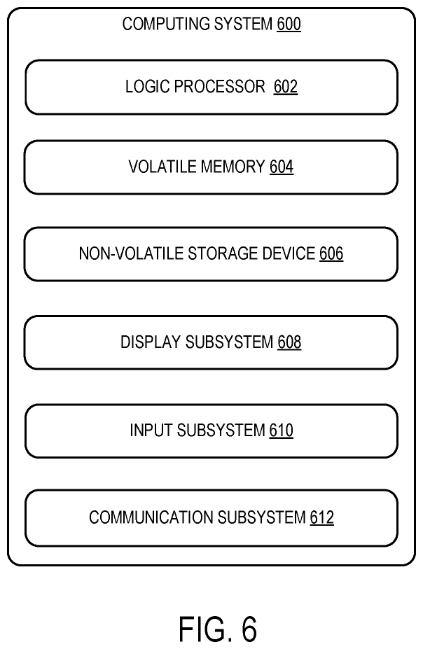

FIG. 6 schematically shows a non-limiting embodiment of a computing system 600 that can enact one or more of the methods and processes described above. Computing system 600 is shown in simplified form. Computing system 600 may take the form of one or more personal computers, server computers, tablet computers, home-entertainment computers, network computing devices, gaming devices, mobile computing devices, mobile communication devices (e.g., smart phone), and/or other computing devices.

Computing system 600 includes a logic processor 602 volatile memory 604, and a non-volatile storage device 606. Computing system 600 may optionally include a display sub system 608, input sub system 610, communication sub system 612, and/or other components not shown in FIG. 6.

Logic processor 602 includes one or more physical devices configured to execute instructions. For example, the logic processor 602 may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor 602 may include one or more physical processors (hardware) configured to execute software instructions. Additionally or alternatively, the logic processor 602 may include one or more hardware logic circuits or firmware devices configured to execute hardware-implemented logic or firmware instructions. Processors of the logic processor 602 may be single-core or multi-core, and the instructions executed thereon may be configured for sequential, parallel, and/or distributed processing. Individual components of the logic processor optionally may be distributed among two or more separate devices, which may be remotely located and/or configured for coordinated processing. Aspects of the logic processor 602 may be virtualized and executed by remotely accessible, networked computing devices configured in a cloud-computing configuration. In such a case, these virtualized aspects are run on different physical logic processors of various different machines, it will be understood.

Volatile memory 604 may include physical devices that include random access memory. Volatile memory 604 is typically utilized by logic processor 602 to temporarily store information during processing of software instructions. It will be appreciated that volatile memory 604 typically does not continue to store instructions when power is cut to the volatile memory 604.

Non-volatile storage device 606 includes one or more physical devices configured to hold instructions executable by the logic processors 602 to implement the methods and processes described herein. When such methods and processes are implemented, the state of non-volatile storage device 606 may be transformed—e.g., to hold different data.

Non-volatile storage device 606 may include physical devices that are removable and/or built-in. Non-volatile storage device 606 may include optical memory (e.g., CD, DVD, HD-DVD, Blu-Ray Disc, etc.), semiconductor memory (e.g., ROM, EPROM, EEPROM, FLASH memory, etc.), and/or magnetic memory (e.g., hard-disk drive, floppy-disk drive, tape drive, MRAM, etc.), or other mass storage device technology. Non-volatile storage device 606 may include nonvolatile, dynamic, static, read/write, read-only, sequential-access, location-addressable, file-addressable, and/or content-addressable devices. It will be appreciated that non-volatile storage device 606 is configured to hold instructions even when power is cut to the non-volatile storage device 606.

Aspects of logic processor 602, volatile memory 604, and non-volatile storage device 606 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The term “program” may be used to describe an aspect of computing system 600 typically implemented in software by a processor to perform a particular function using portions of volatile memory, which function involves transformative processing that specially configures the processor to perform the function. Thus, a program may be instantiated via logic processor 602 executing instructions held by non-volatile storage device 606, using portions of volatile memory 604. It will be understood that different programs may be instantiated from the same application, service, code block, object, library, routine, API, function, etc. Likewise, the same program may be instantiated by different applications, services, code blocks, objects, routines, APIs, functions, etc. The term “program” may encompass individual or groups of executable files, data files, libraries, drivers, scripts, database records, etc.

When included, display subsystem 608 may be used to present a visual representation of data held by non-volatile storage device 606. The visual representation may take the form of a graphical user interface (GUI). As the herein described methods and processes change the data held by the non-volatile storage device, and thus transform the state of the non-volatile storage device, the state of display subsystem 608 may likewise be transformed to visually represent changes in the underlying data. Display subsystem 608 may include one or more display devices utilizing virtually any type of technology. Such display devices may be combined with logic processor 602, volatile memory 604, and/or non-volatile storage device 606 in a shared enclosure, or such display devices may be peripheral display devices.

When included, input subsystem 610 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor.

When included, communication subsystem 612 may be configured to communicatively couple various computing devices described herein with each other, and with other devices. Communication subsystem 612 may include wired and/or wireless communication devices compatible with one or more different communication protocols. As non-limiting examples, the communication subsystem may be configured for communication via a wireless telephone network, or a wired or wireless local- or wide-area network, such as a HDMI over Wi-Fi connection. In some embodiments, the communication subsystem may allow computing system 600 to send and/or receive messages to and/or from other devices via a network such as the Internet.

Another example provides a computing system comprising a processor, and memory comprising instructions executable by the processor to receive a chemical structure input, obtain retrosynthetic step data based on the chemical structure input, determine a chemical structure of a primary chemical in the retrosynthetic step data, the primary chemical being a chemical used as a starting material in a retrosynthetic step, when the structure of the primary chemical is not available in a life-cycle inventory (LCI) database, input the primary chemical into a trained proxy chemical selection model to select a proxy chemical for which an LCI is available and obtain proxy chemical LCI data to include in an estimated LCI for a life cycle assessment (LCA). In some such examples, the trained proxy chemical selection model comprises an artificial neural network (ANN) that is trained with LCI data contained in a plurality of LCIs. In some such examples, the LCI data alternatively or additionally includes, for each of the plurality of LCIs, a chemical structure of the proxy chemical, and the LCI data alternatively or additionally is clustered in the trained proxy chemical selection model based at least upon the chemical structure of the proxy chemical. In some such examples, the instructions alternatively or additionally are executable to, when the structure of the primary chemical is not available in the life-cycle inventory (LCI) database, obtain retrosynthetic step data based on the chemical structure of the primary chemical, and based upon the retrosynthetic step data, determine a chemical structure of an additional primary chemical. In some such examples, the instructions are alternatively or additionally further executable to determine a chemical structure of an ancillary chemical in the retrosynthetic step data, and when the structure of the ancillary chemical is not available in the LCI database, input the ancillary chemical into the trained proxy chemical selection model to obtain a proxy chemical for which an LCI is available, and obtain proxy chemical LCI data to include in the estimated LCI for the LCA. In some such examples, the instructions alternatively or additionally are further executable to store the estimated LCI and also store metadata for the estimated LCI, the metadata comprising information regarding an uncertainty of the estimated LCI. In some such examples, the instructions alternatively or additionally are further executable to, when the structure of the ancillary chemical is available in the LCI database, obtain chemical LCI data to include in the estimated LCI for the LCA.

Another example provides a method for generating an estimated life cycle inventory (LCI) for including in a life-cycle assessment (LCA), the method comprising receiving a chemical structure input, obtaining retrosynthetic step data based on the chemical structure input, determining a chemical structure of a primary chemical in the retrosynthetic step data, the primary chemical being a chemical used as a starting material in a retrosynthetic step, and obtaining proxy chemical LCI data to include in the estimated LCI. In some such examples, the method alternatively or additionally further comprises, when the structure of the primary chemical is not available in a life-cycle inventory (LCI) database, inputting the primary chemical into a trained proxy chemical selection model to select a proxy chemical for which an LCI is available. In some such examples, the LCI data alternatively or additionally includes, for each of the plurality of LCIs, a chemical structure of the proxy chemical, and the LCI data alternatively or additionally is clustered in the trained proxy chemical selection model based at least upon the chemical structure of the proxy chemical. In some such examples, the method alternatively or additionally comprises, when the structure of the primary chemical is not available in the LCI database, obtaining retrosynthetic step data based on the chemical structure of the primary chemical, and based upon the retrosynthetic step data, determining a chemical structure of an additional primary chemical. In some such examples, the method further comprising determining a chemical structure of an ancillary chemical in the retrosynthetic step data. In some such examples, the method alternatively or additionally comprises, when the structure of the ancillary chemical is not available in the LCI database, inputting the ancillary chemical into a trained proxy chemical selection model to obtain a proxy chemical for which an LCI is available, obtaining proxy chemical LCI data to include in the life cycle assessment. In some such examples, the method alternatively or additionally further comprises, when the structure of the ancillary chemical is available in the LCI database, obtaining chemical LCI data to include in the life cycle assessment.

Another example provides a computing system, comprising a processor, and memory comprising instructions executable by the processor to receive a chemical structure input, obtain retrosynthetic step data based on the chemical structure input, identify a chemical transformation in the retrosynthetic step data, retrieve life-cycle inventory (LCI) data associated with the chemical transformation, and include the LCI data in an estimated LCI for a life cycle assessment (LCA). In some such examples, the instructions alternatively or additionally are executable to retrieve LCI data by a trained transformation estimation model. In some such examples, the transformation estimation model alternatively or additionally comprises an artificial neural network (ANN) that is trained with LCI data contained in a plurality of LCIs. In some such examples, the LCI data alternatively or additionally comprises at least a starting material, a primary product, and an energy input. In some such examples, the LCI data alternatively or additionally further comprises a reaction representation, the reaction representation being determined based upon a difference between the starting material and the primary product. In some such examples, the LCI data alternatively or additionally is clustered in the transformation estimation model based at least upon the reaction representation.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system comprising:
a processor, and
memory comprising instructions executable by the processor to:
receive a chemical structure input;
obtain retrosynthetic step data based on the chemical structure input;
determine a chemical structure of a primary chemical in the retrosynthetic step data, the primary chemical being a chemical used as a starting material in a retrosynthetic step;
when the structure of the primary chemical is not available in a life-cycle inventory (LCI) database, input the primary chemical into a trained proxy chemical selection model to select a proxy chemical for which an LCI is available; and
obtain proxy chemical LCI data to include in an estimated LCI for a life cycle assessment (LCA).

2. The computing system of claim 1 wherein, the trained proxy chemical selection model comprises an artificial neural network (ANN) that is trained with LCI data contained in a plurality of LCIs.

3. The computing system of claim 2, wherein
the LCI data includes, for each of the plurality of LCIs, a chemical structure of the proxy chemical; and
the LCI data is clustered in the trained proxy chemical selection model based at least upon the chemical structure of the proxy chemical.

4. The computing system of claim 1, wherein the instructions are further executable to, when the structure of the primary chemical is not available in the life-cycle inventory (LCI) database,
obtain retrosynthetic step data based on the chemical structure of the primary chemical; and
based upon the retrosynthetic step data, determine a chemical structure of an additional primary chemical.

5. The computing system of claim 1, wherein the instructions are further executable to determine a chemical structure of an ancillary chemical in the retrosynthetic step data, and when the structure of the ancillary chemical is not available in the LCI database,
input the ancillary chemical into the trained proxy chemical selection model to obtain a proxy chemical for which an LCI is available; and
obtain proxy chemical LCI data to include in the estimated LCI for the LCA.

6. The computing system of claim 1, wherein the instructions are further executable to store the estimated LCI and also store metadata for the estimated LCI, the metadata comprising information regarding an uncertainty of the estimated LCI.

7. The computing system of claim 5, wherein the instructions are further executable to, when the structure of the ancillary chemical is available in the LCI database, obtain chemical LCI data to include in the estimated LCI for the LCA.

8. A method for generating an estimated life cycle inventory (LCI) for including in a life-cycle assessment (LCA), the method comprising:
receiving a chemical structure input;
obtaining retrosynthetic step data based on the chemical structure input;
determining a chemical structure of a primary chemical in the retrosynthetic step data, the primary chemical being a chemical used as a starting material in a retrosynthetic step;
and
obtaining proxy chemical LCI data to include in the estimated LCI.

9. The method of claim 8, further comprising, when the structure of the primary chemical is not available in a life-cycle inventory (LCI) database, inputting the primary chemical into a trained proxy chemical selection model to select a proxy chemical for which an LCI is available.

10. The method of claim 9, wherein
the LCI data includes, for each of the plurality of LCIs, a chemical structure of the proxy chemical; and
the LCI data is clustered in the trained proxy chemical selection model based at least upon the chemical structure of the proxy chemical.

11. The method of claim 8, further comprising, when the structure of the primary chemical is not available in the LCI database,
obtaining retrosynthetic step data based on the chemical structure of the primary chemical; and
based upon the retrosynthetic step data, determining a chemical structure of an additional primary chemical.

12. The method of claim 8, further comprising determining a chemical structure of an ancillary chemical in the retrosynthetic step data.

13. The method of claim 12, further comprising, when the structure of the ancillary chemical is not available in the LCI database:
inputting the ancillary chemical into a trained proxy chemical selection model to obtain a proxy chemical for which an LCI is available; and
obtaining proxy chemical LCI data to include in the life cycle assessment.

14. The method of claim 12, further comprising, when the structure of the ancillary chemical is available in the LCI database, obtaining chemical LCI data to include in the life cycle assessment.

15. A computing system, comprising:
a processor, and
memory comprising instructions executable by the processor to:
receive a chemical structure input;
obtain retrosynthetic step data based on the chemical structure input;
identify a chemical transformation in the retrosynthetic step data;
retrieve life-cycle inventory (LCI) data associated with the chemical transformation; and
include the LCI data in an estimated LCI for a life cycle assessment (LCA).

16. The computing system of claim 15 wherein the instructions are executable to retrieve LCI data by a trained transformation estimation model.

17. The computing system of claim 16, wherein the transformation estimation model comprises an artificial neural network (ANN) that is trained with LCI data contained in a plurality of LCIs.

18. The computing system of claim 17, wherein the LCI data comprises at least a starting material, a primary product, and an energy input.

19. The computing system of claim 18, wherein the LCI data further comprises a reaction representation, the reaction representation being determined based upon a difference between the starting material and the primary product.

20. The computing system of claim 19, wherein the LCI data is clustered in the transformation estimation model based at least upon the reaction representation.
